(19) United States
(12) Patent Application Publication
(10) Pub. No.: US 2014/0071072 A1
(43) Pub. Date: Mar. 13, 2014

(54) MEDICAL IMAGE DISPLAY APPARATUS, METHOD AND PROGRAM

(71) Applicant: FUJIFILM Corporation, Tokyo (JP)

(72) Inventor: Yoshinori ITAI, Tokyo (JP)

(73) Assignee: FUJIFILM Corporation, Tokyo (JP)

(21) Appl. No.: 14/020,713

(22) Filed: Sep. 6, 2013

(30) Foreign Application Priority Data
Sep. 12, 2012 (JP) 2012-200428

(51) Int. Cl. A61B 19/00

(52) U.S. Cl. CPC A61B 19/50
USPC 345/173

(57) ABSTRACT

A display screen displaying a three-dimensional medical image of a subject to be examined, an operation detection unit receiving an operation input by detecting a touch on the display screen, and a processing setting unit in which a processing table has been set in advance are provided. The processing table links a series of operation inputs to be received on the display screen and kinds of processing to be performed on the image in such a manner that the serial positions of the inputs correspond to the kinds of processing. Further, an image processing unit that performs, on the image, processing corresponding to the serial position of the operation input with reference to the processing table when the operation input has been received on the display screen, and a display control unit that displays, on the display screen, the three-dimensional medical image on which processing has been performed are provided.

### BACKGROUND OF THE INVENTION

1. Field of the Invention

The present invention relates to a medical image display apparatus, method and program for displaying a three-dimensional medical image of a subject to be examined. In particular, the present invention relates to a medical image display apparatus, method and program for displaying a three-dimensional medical image used in surgery simulation or the like, and for receiving an operation input by a user on the displayed three-dimensional medical image.

2. Description of the Related Art

Conventionally, a three-dimensional medical image of a subject to be examined was obtained by imaging the subject to be examined, for example, by using a CT (Computed Tomography) apparatus, an MRI (Magnetic Resonance Imaging) apparatus, or the like. The obtained three-dimensional medical image was displayed on a display by using a display method, such as volume rendering, and presented to a user.

Further, in recent years, a surgery simulation method using a three-dimensional medical image displayed by volume rendering as described above was proposed.

For example, Japanese Unexamined Patent Publication No. 2008-134373 (Patent Document 1) proposes an apparatus that can give a sensation similar to an actual surgery by generating a simulation living body by setting physical properties on a three-dimensional medical image, and also by measuring a movement amount of a surgical instrument by making a surgeon actually operate the surgical instrument in the real world. Reaction force corresponding to the location of the surgical instrument operated by the surgeon and the location of contact with the simulation living body is applied to an operator to give such a sensation.

### SUMMARY OF THE INVENT ION

However, the surgery simulation apparatus disclosed in Patent Document 1 is too large and complex for application, and an operation is not easy. Therefore, there is a demand for a surgery simulation apparatus having simpler structure, and which can be used at any place in medical facilities, such as a hospital.

Further, as the aforementioned surgery simulation method using a three-dimensional medical image, for example, a user may specify a predetermined region in the three-dimensional medical image by a mouse or the like while observing the three-dimensional medical image displayed on a display. Further, deformation processing, cut processing or the like may be performed on the three-dimensional medical image at the location specified by the user.

However, if a location in a three-dimensional medical image must be specified by a mouse or the like, as described above, and further processing to be performed on the location must be selected, it is necessary to specify a location on the three-dimensional medical image by moving the mouse each time, and to select processing to be performed on the location with respect to each surgery action. Therefore, the operation characteristics are not good, and the operation is troublesome. Further, although both hands are often used in an actual surgery, only one input is receivable at one time when a mouse is used in input as described above. Therefore, only one surgery action is simulatable at one time, and it is impossible to simulate a surgery action performed by both hands.

Meanwhile, Japanese Unexamined Patent Publication No. 2000-222130 (Patent Document 2) proposes simultaneously receiving inputs on a touch panel performed by plural fingers, and performing predetermined processing based on the received content. However, Patent Document 2 fails to propose anything about the aforementioned surgery simulation.

In view of the foregoing circumstances, it is an object of the present invention to provide a medical image display apparatus having simple structure, and which can be carried to any place to perform surgery simulation, and which can simulate a surgery action performed by both hands. Further, it is another object of the present invention to provide such a medical image display method and program.

A medical image display apparatus of the present invention is a medical image display apparatus comprising:

a display operation receiving unit including a display screen that displays a three-dimensional medical image of a subject to be examined and an operation detection unit that receives an operation input by detecting a touch on the display screen;

a processing setting unit in which a processing table has been set in advance, and the processing table linking a series of operation inputs to be received by the display operation receiving unit and kinds of processing to be performed on the three-dimensional medical image in such a manner that the serial positions of the series of operation inputs correspond to the kinds of processing, respectively;

an image processing unit that performs, on the three-dimensional medical image, processing corresponding to the serial position of the operation input with reference to the processing table when the display operation receiving unit has received the operation input; and

a display control unit that displays, on the display screen, the three-dimensional medical image on which processing has been performed by the image processing unit.

In the medical image display apparatus, the image processing unit may regard a plurality of operation inputs as operation inputs performed at the same serial position when the display operation receiving unit received the plurality of operation inputs within a time period that had been set in advance, and the image processing unit may perform processing corresponding to the same serial position on the three-dimensional medical image.

As the processing to be performed on the three-dimensional medical image, at least one of rotation processing, parallel translation processing, deformation processing, cut processing, deletion processing and marking processing may be used.

The processing setting unit may include a plurality of kinds of processing tables, and the image processing unit may perform processing on the three-dimensional medical image with reference to a selected one of the plurality of kinds of processing tables.

The display control unit may display, on the display screen, a selection screen for selecting one of the plurality of kinds of processing tables.

The display control unit may display icons corresponding to the plurality of kinds of processing tables, respectively, on the display screen.

As the deformation processing, a non-rigid body deformation processing may be used.

Further, an image obtainment unit that obtains the three-dimensional medical image of a living body and an image extraction unit that extracts a three-dimensional medical image of an anatomical tissue from the three-dimensional medical image of the living body may be provided. The image processing unit may perform processing on the three-dimensional medical image of the anatomical tissue.

The anatomical tissue may be one of a head, a lung or lungs, a liver, a large intestine and a blood vessel or vessels.

A medical image display method of the present invention is a medical image display method comprising the steps of:

displaying a three-dimensional medical image of a subject to be examined on a display screen;

receiving an operation input by detecting a touch on the display screen;

performing, on the three-dimensional medical image, processing corresponding to the received operation input; and

displaying, on the display screen, the three-dimensional medical image on which processing has been performed,

wherein a processing table linking a series of operation inputs to be received and kinds of processing to be performed on the three-dimensional medical image in such a manner that the serial positions of the series of operation inputs correspond to the kinds of processing respectively has been set in advance, and

wherein processing corresponding to the serial position of the operation input is performed on the three-dimensional medical image with reference to the processing table when the operation input has been received, and

wherein the three-dimensional medical image on which processing has been performed is displayed on the display screen.

A medical image display program of the present invention is a medical image display program for causing a computer to execute procedures of:

displaying a three-dimensional medical image of a subject to be examined on a display screen;

receiving an operation input by detecting a touch on the display screen;

performing, on the three-dimensional medical image, processing corresponding to the received operation input; and

displaying, on the display screen, the three-dimensional medical image on which processing has been performed,

wherein a procedure of referring to a processing table linking a series of operation inputs to be received and kinds of processing to be performed on the three-dimensional medical image in such a manner that the serial positions of the series of operation inputs correspond to the kinds of processing respectively when the operation input has been received,

a procedure of performing, on the three-dimensional medical image, processing corresponding to the serial position of the operation input with reference to the processing table, and

a procedure of displaying, on the display screen, the three-dimensional medical image on which processing has been performed are executed.

According to the medical image display apparatus, method and program of the present invention, a display operation receiving unit including a display screen that displays a three-dimensional medical image of a subject to be examined and an operation detection unit that receives an operation input by detecting a touch on the display screen is used. Further, a processing table linking a series of operation inputs to be received by the display operation receiving unit and kinds of processing to be performed on the three-dimensional medical image in such a manner that the serial positions of the series of operation inputs correspond to the kinds of processing respectively has been set in advance. When the display operation receiving unit has received the operation input, processing corresponding to the serial position of the operation input is performed on the three-dimensional medical image with reference to the processing table, and the three-dimensional medical image on which processing has been performed is displayed on the display screen. Therefore, if a user performs just a predetermined operation input on the display screen of the display operation receiving unit, processing based on the content and the serial position of the operation input is performed on the three-dimensional medical image, and the processed three-dimensional medical image can be displayed.

Unlike the aforementioned techniques, a position on a three-dimensional medical image does not need to be specified by a mouse and the content of processing to be performed on the position does not need to be selected for each surgery action. Therefore, it is possible to easily perform surgery simulation.

For example, if a tablet terminal including a touch panel is used, as the display operation receiving unit as described above, it is possible to receive operation inputs performed by both hands. Therefore, it is possible to simultaneously perform, on the three-dimensional medical image, processing based on the operation inputs performed by both hands. Hence, simulation is possible while feeling a sensation close to an actual surgery.

Further, when plural kinds of processing tables, as described above, are set, and processing is performed on a three-dimensional medical image with reference to the selected one of the plural kinds of processing tables, it is possible to increase the variation of surgery simulation.

Further, an icon corresponding to each of plural kinds of processing tables may be displayed on a display screen, and one of the processing tables may be selected by selection of an icon. In such a case, it is possible to select the processing table through an easier operation.

Note that the program of the present invention may be provided being recorded on a computer readable medium. Those who are skilled in the art would know that computer readable media are not limited to any specific type of device, and include, but are not limited to: floppy disks, CD's, RAM's, ROM's, hard disks, magnetic tapes, and internet downloads, in which computer instructions can be stored and/or transmitted. Transmission of the computer instructions through a network or through wireless transmission means is also within the scope of this invention. Additionally, computer instructions include, but are not limited to: source, object and executable code, and can be in any language including higher level languages, assembly language, and machine language.

### BRIEF DESCRIPTION OF THE DRAWINGS

FIG. 1 is a perspective view illustrating the external view of a tablet terminal using an embodiment of a medical image display apparatus of the present invention;

FIG. 2 is a block diagram illustrating the internal configuration of the tablet terminal illustrated in FIG. 1;

FIG. 3 is a diagram illustrating an example of a processing table;

FIG. 4 is a flow chart for explaining the action of a tablet terminal using an embodiment of the medical image display apparatus of the present invention;

FIG. 5 is a diagram for explaining an example of surgery simulation using the processing table illustrated in FIG. 3;

FIG. 6 is a diagram for explaining an example of non-rigid body deformation processing by performing an operation input with two fingers;

FIG. 7 is a diagram illustrating another example of a processing table;

FIG. 8 is a diagram for explaining an example of surgery simulation using the processing table illustrated in FIG. 7;

FIG. 9 is a diagram illustrating another example of a processing table; and

FIG. 10 is a diagram illustrating an example in which icons corresponding to plural kinds of processing tables are displayed.

### DESCRIPTION OF THE PREFERRED EMBODIMENTS

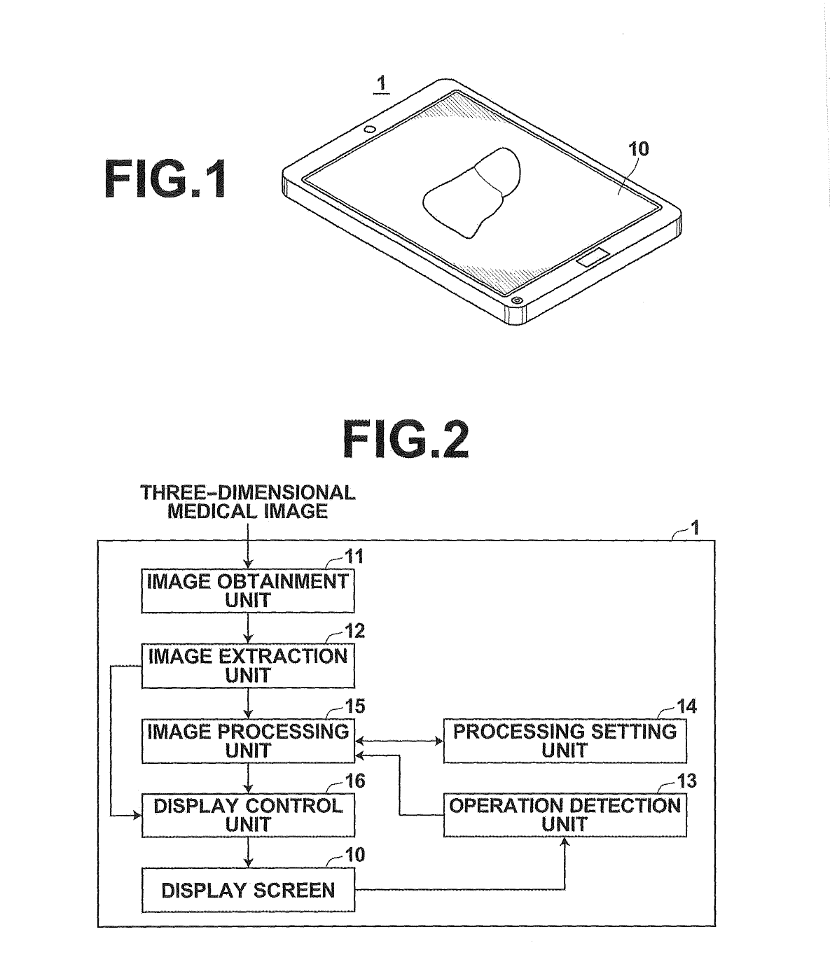

Hereinafter, an embodiment of a medical image display apparatus, method and program of the present invention will be described in detail with reference to drawings. FIG. 1 is a perspective view of a tablet terminal including an embodiment of a medical image display apparatus of the present invention. FIG. 2 is a block diagram illustrating the internal configuration of the tablet terminal illustrated in FIG. 1.

As illustrated in FIG. 1, a tablet terminal 1 according to an embodiment of the present invention includes a display screen 10 for displaying an input three-dimensional medical image of a subject to be examined. The display screen 10 is a liquid crystal screen constituting a touch panel, which is touched by a user to perform a predetermined operation input. A user's touch on the display screen 10 is detected by an operation detection unit 13, which will be described later, and an operation input by the user is received by detecting the touch. The display screen 10 and the operation detection unit 13 constitute a display operation receiving unit recited in the claims of the present application.

The tablet terminal 1 according to an embodiment of the present invention is a terminal in which an embodiment of a medical image display program of the present invention has been installed. The medical image display program is stored in a recording medium, such as a DVD and a CD-ROM, or a server computer or the like that is accessible from the outside, and which is connected to a network. The medical image display program is read out from the recording medium, the server computer or the like based on a request by a doctor or the like, and downloaded and installed in the tablet terminal 1.

The tablet terminal 1 according to the embodiment of the present invention includes a central processing unit (CPU), a semiconductor memory, and a storage device, such as a hard disk, in which the medical image display program has been installed. These kinds of hardware constitute an image obtainment unit 11, an image extraction unit 12, the operation detection unit 13, a processing setting unit 14, an image processing unit 15, and a display control unit 16, as illustrated in FIG. 2. Each of the units functions when the medical image display program installed in the hard disk is executed by the central processing unit.

The image obtainment unit 11 obtains a three-dimensional medical image of a subject to be examined that has been imaged in advance. Specifically, the image obtainment unit 11 obtains a three-dimensional medical image obtained by imaging the subject to be examined in a CT examination, an MRI examination or the like. Such a three-dimensional medical image has been stored in advance in a data server or the like, and the three-dimensional medical image is obtained by connecting the data server and the tablet terminal 1 to each other through a wireless or wire connection.

The image extraction unit 12 extracts a three-dimensional medical image of an anatomical tissue from the three-dimensional medical image obtained by the image obtainment unit 11. In the embodiment of the present invention, a three-dimensional medical image of a liver is extracted. Alternatively, a head, a lung or lungs, a large intestine, a blood vessel or vessels, or the like may be extracted. Since there are already known techniques for extracting these anatomical tissues, detailed descriptions will be omitted.

The operation detection unit 13 includes a sensor for detecting a user's touch on the display screen 10. The operation detection unit 13 receives, based on the detection result by the sensor, a user's operation input. For example, a drag operation, a drag and drop operation, a tap operation and the like may be received, as operation inputs, at the operation detection unit 13. Alternatively, other general operation inputs at a touch panel may be received.

A processing table is set in advance in the processing setting unit 14. The processing table links a series of operation inputs to be received by the display operation receiving unit 13 and kinds of processing to be performed on the three-dimensional medical image in such a manner that the serial positions of the series of operation inputs correspond to the kinds of processing respectively.

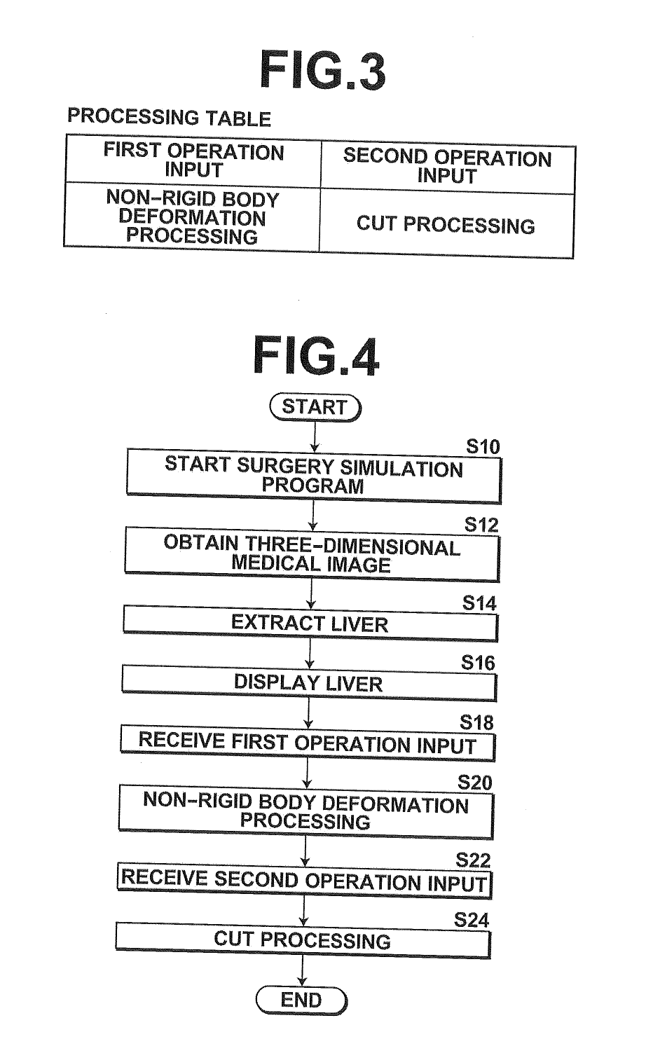

As processing on the three-dimensional medical image, there are rotation processing, parallel translation processing, non-rigid body deformation processing, cut processing, deletion processing, marking processing, and the like. In the embodiment of the present invention, the non-rigid body deformation processing and the cut processing of these kinds of processing are set in the processing table. Specifically, a processing table as illustrated in FIG. 3 is set in advance in the processing setting unit 14. In the processing table, non-rigid body deformation processing is linked with a first operation input, which is a first input, and cut processing is linked with a second operation input, which is a second input. In the embodiment of the present invention, the first operation input is a drag operation, and the second operation input is a drag and drop operation.

Further, the marking processing is, for example, processing for attaching a predetermined marking image to a three-dimensional medical image of a liver. For example, when a user performs a drag and drop operation in such a manner to enclose a tumor region in a three-dimensional medical image of a liver, an image of a circle or an ellipse is attached to the enclosed range. Alternatively, when the user performs a drag and drop operation on a position to be cut in the three-dimensional medical image of the liver, an image of a line is attached to the position at which the drag operation has been performed.

When the operation detection unit 13 has received an operation input, the image processing unit 15 refers to the processing table set in the processing setting unit 14, and performs, on the three-dimensional medical image, processing corresponding to the serial position of the operation input. Specifically, in the embodiment of the present invention, when the operation detection unit 13 has received, as the first operation input, a first drag operation, non-rigid body deformation processing is performed on the three-dimensional medical image of the liver extracted by the image extraction unit 12 based on the direction and the amount of the drag operation. Then, when the operation detection unit 13 has received, as the second operation input, a second drag and drop operation, cut processing based on the direction and the amount of the drag operation is performed on the three-dimensional medical image of the liver on which non-rigid body deformation processing has been performed. The non-rigid body deformation processing and the cut processing will be described later in detail.

The display control unit 16 displays, on the display screen 10, the three-dimensional image of the liver extracted by the image extraction unit 12. After then, the display control unit 16 displays, on the display screen 10, the three-dimensional medical image of the liver on which the non-rigid body deformation processing and the cut processing have been performed at the image processing unit 15 based on the operation inputs by the user.

Next, the action of the tablet terminal 1 of the embodiment of the present invention will be described with reference to a flow chart illustrated in FIG. 4.

First, an icon or the like displayed on the display screen 10 of the tablet terminal 1 is tapped, and a surgery simulation program is started. Accordingly, the medical image display program according to the embodiment of the present invention is started (S10).

Next, the user inputs an instruction to obtain a three-dimensional medical image of a subject to be examined. Accordingly, the three-dimensional medical image of the subject to be examined is read out from a data server or the like, and obtained by the image obtainment unit 11 (S12).

The three-dimensional medical image obtained by the image obtainment unit 11 is output to the image extraction unit 12. The image extraction unit 12 extracts a three-dimensional medical image of the liver from the received three-dimensional medical image (S14).

The three-dimensional medical image of the liver extracted by the image extraction unit 12 is output to the display control unit 16, and the display control unit 16 displays the three-dimensional medical image of the liver on the display screen 10 of the tablet terminal 1 (S16). Here, a three-dimensional medical image of blood vessels in the vicinity of the liver may be separately extracted besides the liver, and the three-dimensional medical image of the blood vessels may be displayed together with the three-dimensional medical image of the liver.

Then, first, the user touches a desirable point on the liver in the three-dimensional medical image of the liver displayed on the display screen 10. After then, a drag operation is performed, and this drag operation is received, as a first operation input, by the operation detection unit 13 (S18).

When the operation detection unit 13 receives the first operation input, the image processing unit 15 refers to the processing table set in the processing setting unit 14, and performs non-rigid body deformation processing, which corresponds to the first operation input, on the three-dimensional medical image of the liver (S20).

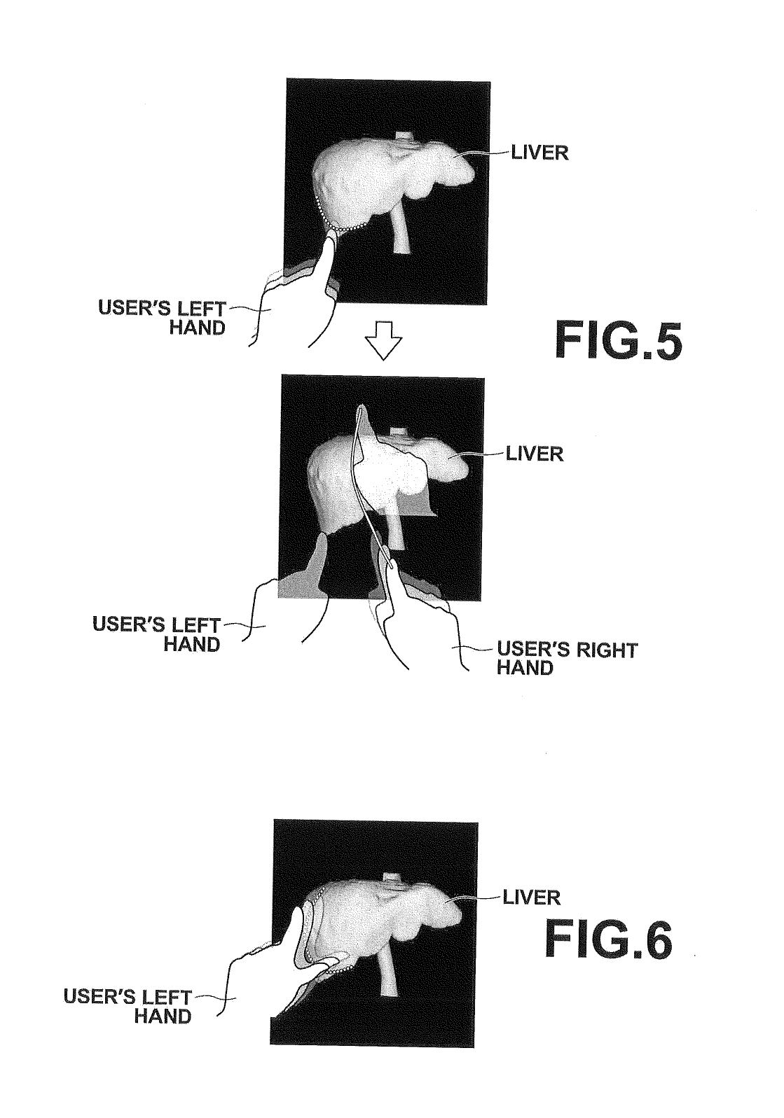

Specifically, for example, as illustrated in the upper section of FIG. 5, when the user first touches the bottom edge of the liver with a finger of his/her left hand, and drags the finger downward, non-rigid body deformation processing is performed on the three-dimensional medical image of the liver in such a manner that the bottom edge of the liver is pulled and stretched downward. Specifically, the non-rigid body deformation processing is performed in the following manner. First, control points are evenly arranged in the three-dimensional medical image of the liver, and one of the control points in the vicinity of a point touched by the user is used as a control point of interest. Then, the control point of interest is moved to a position to which the user has dragged his/her finger. Further, control points in the vicinity of the control point of interest are moved in a similar manner to the control point of interest. Then, non-rigid body deformation processing is performed on the three-dimensional medical image of the liver based on the position information about the control points that have been moved, and the three-dimensional medical image of the liver after non-rigid body deformation processing is generated. Here, known non-rigid body deformation in image registration may be used for non-rigid body deformation processing. As non-rigid body deformation processing in image registration, techniques disclosed, for example, in W. R. CRUM et al., “Non-rigid image registration: theory and practice”, The British Journal of Radiology, Vol. 77, pp. S140-S153, 2004 and the like may be used.

Next, as illustrated in the lower section of FIG. 5, a drag and drop operation by the right hand of the user is performed on the display screen 10 in which the three-dimensional medical image of the liver after non-rigid body deformation processing has been performed is displayed. This operation is received, as a second operation input, by the operation detection unit 13 (S22). Here, the left hand of the user keeps pulling the bottom edge of the liver. In other words, it is possible to receive the second operation input in the state in which the first operation input has been received.

When the operation detection unit 13 has received the second operation input, the image processing unit 15 refers to the processing table set in the processing setting unit 14, and performs, on the three-dimensional medical image of the liver, cut processing corresponding to the second operation input (S24).

Then, the display control unit 16 displays, on the display screen 10, the three-dimensional medical image of the liver on which cut processing has been performed by the image processing unit 15. Here, for example, when the liver has been cut into two sections, the three-dimensional medical image of the liver after cut processing may be displayed in such a manner that one of the sections has been deleted. Alternatively, the three-dimensional medical image may be displayed in such a manner that a space is provided between the two sections. Alternatively, the three-dimensional medical image of the liver after cut processing may be displayed in such a manner that one of the sections is deleted, but only an image of blood vessels of the deleted liver section is displayed.

According to the tablet terminal 1 of the aforementioned embodiment, the processing table linking a series of operation inputs to be received by the tablet terminal 1 and kinds of processing to be performed on the three-dimensional medical image in such a manner that the serial positions of the series of operation inputs correspond to the kinds of processing respectively is set in advance. When an operation input is received at the tablet terminal 1, processing corresponding to the serial position of the operation input is performed on the three-dimensional medical image with reference to the processing table. Further, the three-dimensional medical image on which the processing has been performed is displayed on the display screen 10. Therefore, it is possible to perform, on the three-dimensional medical image, processing corresponding to the content and the serial position of an operation input if the user performs just the predetermined operation input on the display screen 10 of the tablet terminal 1, and to display the three-dimensional medical image after processing.

Therefore, it is not necessary to specify a position on the three-dimensional medical image by a mouse, and to select the content of processing to be performed on the position, as described above, for each surgery action. Therefore, easy surgery simulation is possible.

Further, since it is possible to receive an operation input performed by both hands on the display screen 10 of the tablet terminal 1, it is possible to simultaneously perform, on the three-dimensional medical image, processing corresponding to the operation input. Therefore, it is possible to simulate a surgery in such a manner that a sensation similar to an actual surgery is felt. Further, it is possible to carry the apparatus to any place to simulate a surgery.

In the descriptions of the embodiments, a drag operation with a user's finger was received as the first operation input. However, it is not necessary that the operation is performed by one finger. As illustrated in FIG. 6, a drag operation with two fingers may be received, and non-rigid body deformation processing based on the drag operation with the two fingers may be performed on the three-dimensional medical image of the liver.

When a drag operation with two fingers is received, as described above, if a drag operation with one of the two fingers (for example, a forefinger) and a drag operation with the other finger (for example, a thumb) are performed exactly at the same timing, the image processing unit 15 may recognize the drag operations with the two fingers, as the first operation input. However, for example, if the timing of touch by one of the fingers and the timing of touch by the other finger are not the same, the image processing unit 15 recognizes the drag operation by the first touch, as the first operation input, and recognizes the drag operation by the next touch, as the second operation input. Therefore, processing performed on the three-dimensional medical image differs depending on the fingers used in the input operation.

To prevent such a problem, when plural operation inputs are received by the operation detection unit 13 within a time period that has been set in advance, the image processing unit 15 regards the plural operation inputs, as an operation input at the same serial position, and performs, on the three-dimensional medical image, processing corresponding to the serial position. In other words, when operation input is performed with two fingers, as described above, if the operation input with the first finger and the operation input with the second finger are performed within a time period that has been set in advance, the image processing unit 15 recognizes both of the operation inputs performed by the two fingers, as the first operation input. The image processing unit 15 performs non-rigid body deformation, as processing corresponding to the drag operations performed by the two fingers.

As the time period that has been set in advance, for example, about 0.5 to 1 second may be set. Alternatively, a user may input setting of an arbitrary time period.

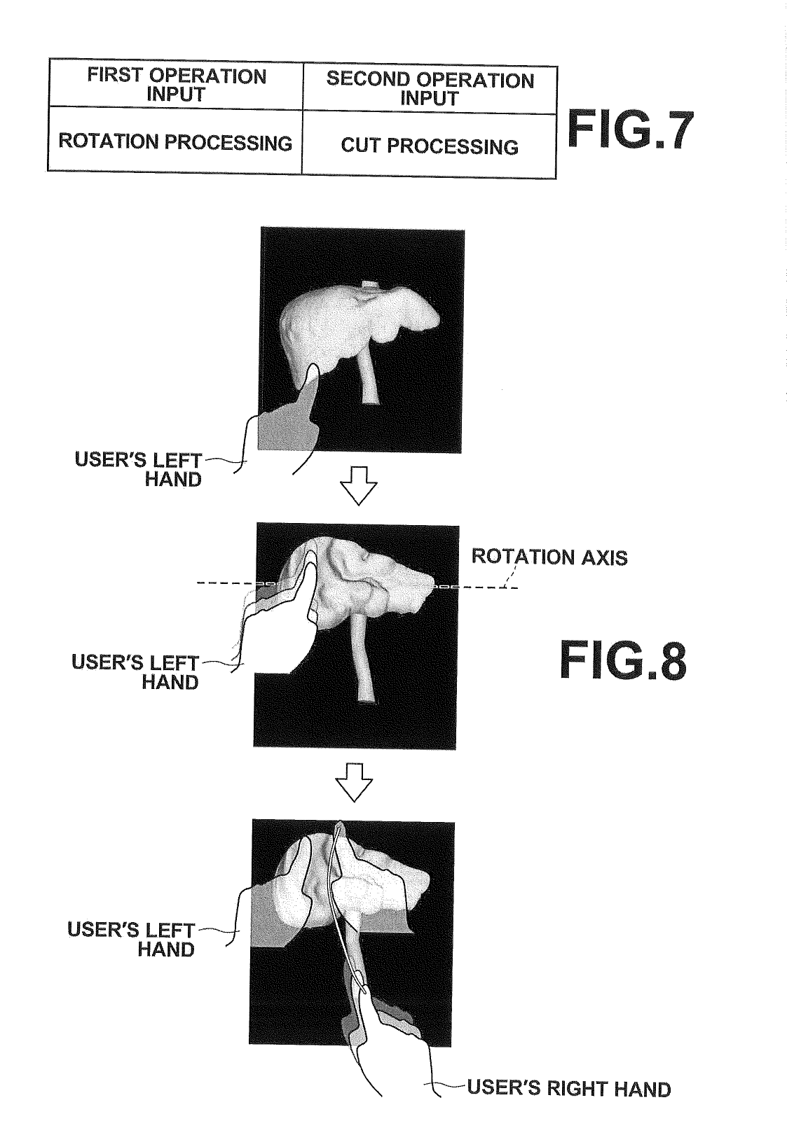

In the aforementioned embodiment, the processing table, as illustrated in FIG. 3, is set in the processing setting unit 14. It is not necessary that the processing table is set in such a manner. Alternatively, a processing table, as illustrated in FIG. 7, may be set. In the processing table illustrated in FIG. 7, rotation processing is set as processing corresponding to the first input operation, and cut processing is set as processing corresponding to the second input operation. Next, an action when the processing table illustrated in FIG. 7 is set in the processing setting unit 14 will be described.

First, as illustrated in FIG. 8, a user touches a desirable point on a liver in a three-dimensional medical image of the liver displayed on the display screen 10. After then, the user performs a drag operation, and the operation detection unit 13 receives this operation, as a first operation input.

When the operation detection unit 13 receives the first operation input, the image processing unit 15 refers to a processing table set in the processing setting unit 14, and performs, on the three-dimensional medical image of the liver, rotation processing corresponding to the first operation input. Specifically, when a upward drag operation is performed as illustrated in FIG. 8, rotation processing is performed on the liver with respect to an axis extending in a horizontal direction in the liver, as the rotation axis. The middle section of FIG. 8 illustrates a diagram in which the posterior side of the liver is visible by the rotation processing. Here, rotation processing when the upward drag operation was performed has been described. However, for example, when a drag operation is performed in the horizontal direction, rotation processing should be performed on the liver with respect to an axis extending in a vertical direction in the liver, as the rotation axis. In other words, rotation processing should be performed on the liver with respect to an axis extending in a direction perpendicular to the direction of drag in the liver, as the rotation axis.

Next, as illustrated in FIG. 8, a drag and drop operation is performed with the right hand of the user on the display screen 10 on which the three-dimensional medical image of the liver after the aforementioned rotation processing is displayed. The operation detection unit 13 receives this operation, as the second operation input.

When the operation detection unit 13 receives the second operation input, the image processing unit 15 refers to the processing table set in the processing setting unit 14, and performs cut processing corresponding to the second operation input on the three-dimensional medical image of the liver. Here, the three-dimensional medical image of the liver after cut processing is displayed as described already.

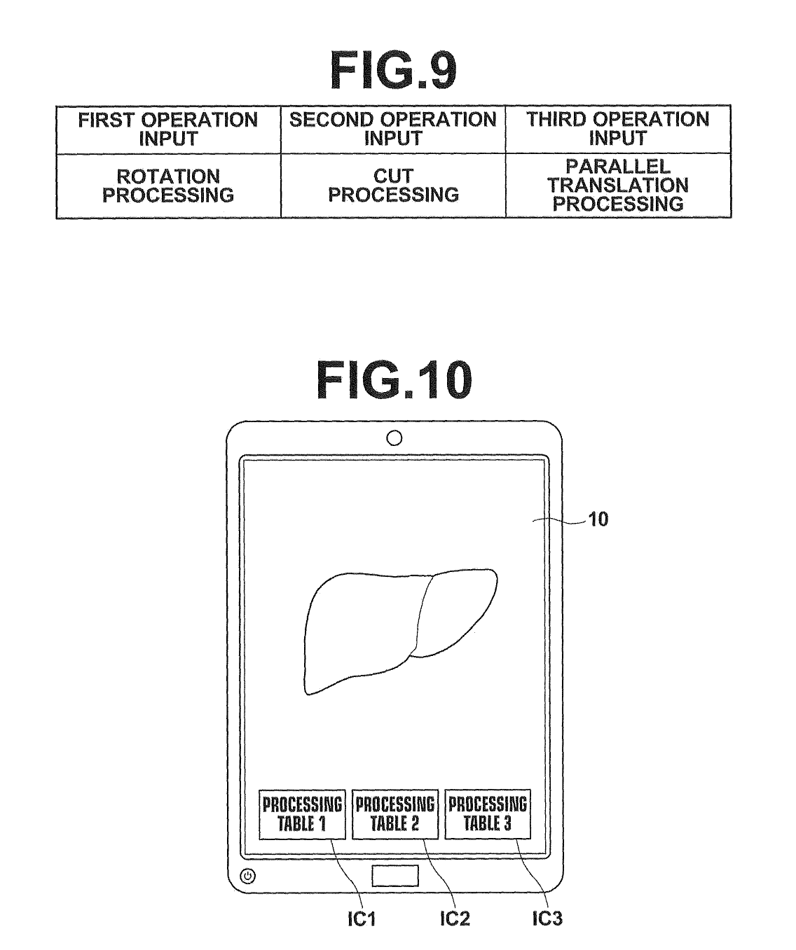

In the aforementioned embodiments, processing corresponding to two operation inputs (first and second operation inputs) is set in the processing table. Alternatively, processing corresponding to three or more input operations may be set, and processing corresponding to each operation input may be performed on the three-dimensional medical image in the order of input of operations. Specifically, for example, as illustrated in FIG. 9, rotation processing may be set as processing corresponding to the first operation input, and cut processing may be set as processing corresponding to the second operation input, and parallel translation processing may be set as processing corresponding to the third operation input. When the processing table illustrated in FIG. 9 has been set, the action till cutting processing, which corresponds to the second operation input, is similar to the aforementioned example. When a drag operation is performed by the user, as the third operation input, one of the two sections of the cut liver on which the drag operation has been performed is moved in parallel in the direction of drag.

In the aforementioned embodiments, a case of setting a processing table was described. Alternatively, plural kinds of processing tables, in which the content of operation inputs and the content of processing are different, may be set. Then, one of the plural kinds of processing tables may be selected by the user, and the image processing unit 15 may perform processing on the three-dimensional medical image by using the selected processing table.

As a method for selecting the processing table, for example, the display control unit 16 should display a selection screen for selecting one of the plural kinds of processing tables on the display screen 10, and a user should select one of the processing tables on the selection screen. As the screen for selecting a processing table, for example, the display control unit 16 may display icons IC1 through IC3 corresponding to plural kinds of processing tables 1 through 3, respectively, on the display screen 10, as illustrated in FIG. 10.

What is claimed is:

1. A medical image display apparatus comprising:
a display operation receiving unit including a display screen that displays a three-dimensional medical image of a subject to be examined and an operation detection unit that receives an operation input by detecting a touch on the display screen;
a processing setting unit in which a processing table has been set in advance, and the processing table linking a series of operation inputs to be received by the display operation receiving unit and kinds of processing to be performed on the three-dimensional medical image in such a manner that the serial positions of the series of operation inputs correspond to the kinds of processing, respectively;
an image processing unit that performs, on the three-dimensional medical image, processing corresponding to the serial position of the operation input with reference to the processing table when the display operation receiving unit has received the operation input; and
a display control unit that displays, on the display screen, the three-dimensional medical image on which processing has been performed by the image processing unit.

2. The medical image display apparatus, as defined in claim 1, wherein the image processing unit regards a plurality of operation inputs as operation inputs performed at the same serial position when the display operation receiving unit received the plurality of operation inputs within a time period that had been set in advance, and the image processing unit performs processing corresponding to the same serial position on the three-dimensional medical image.

3. The medical image display apparatus, as defined in claim 1, wherein the processing to be performed on the three-dimensional medical image is at least one of rotation processing, parallel translation processing, deformation processing, cut processing, deletion processing and marking processing.

4. The medical image display apparatus, as defined in claim 1, wherein the processing setting unit includes a plurality of kinds of processing tables, and
wherein the image processing unit performs processing on the three-dimensional medical image with reference to a selected one of the plurality of kinds of processing tables.

5. The medical image display apparatus, as defined in claim 4, wherein the display control unit displays, on the display screen, a selection screen for selecting one of the plurality of kinds of processing tables.

6. The medical image display apparatus, as defined in claim 5, wherein the display control unit displays icons corresponding to the plurality of kinds of processing tables, respectively, on the display screen.

7. The medical image display apparatus, as defined in claim 3, wherein the deformation processing is a non-rigid body deformation processing.

8. The medical image display apparatus, as defined in claim 1, the apparatus further comprising:
an image obtainment unit that obtains the three-dimensional medical image of a living body; and
an image extraction unit that extracts a three-dimensional medical image of an anatomical tissue from the three-dimensional medical image of the living body,
wherein the image processing unit performs processing on the three-dimensional medical image of the anatomical tissue.

9. The medical image display apparatus, as defined in claim 8, wherein the anatomical tissue is one of a head, a lung or lungs, a liver, a large intestine and a blood vessel or vessels.

10. A medical image display method comprising the steps of:
displaying a three-dimensional medical image of a subject to be examined on a display screen;
receiving an operation input by detecting a touch on the display screen;
performing, on the three-dimensional medical image, processing corresponding to the received operation input; and
displaying, on the display screen, the three-dimensional medical image on which processing has been performed,
wherein a processing table linking a series of operation inputs to be received and kinds of processing to be performed on the three-dimensional medical image in such a manner that the serial positions of the series of operation inputs correspond to the kinds of processing respectively has been set in advance, and
wherein processing corresponding to the serial position of the operation input is performed on the three-dimensional medical image with reference to the processing table when the operation input has been received, and
wherein the three-dimensional medical image on which processing has been performed is displayed on the display screen.

11. A non-transitory computer-readable recording medium storing therein a medical image display program for causing a computer to execute procedures of:
displaying a three-dimensional medical image of a subject to be examined on a display screen;
receiving an operation input by detecting a touch on the display screen;
performing, on the three-dimensional medical image, processing corresponding to the received operation input; and
displaying, on the display screen, the three-dimensional medical image on which processing has been performed,
wherein a procedure of referring to a processing table linking a series of operation inputs to be received and kinds of processing to be performed on the three-dimensional medical image in such a manner that the serial positions of the series of operation inputs correspond to the kinds of processing respectively when the operation input has been received,
a procedure of performing, on the three-dimensional medical image, processing corresponding to the serial position of the operation input with reference to the processing table, and
a procedure of displaying, on the display screen, the three-dimensional medical image on which processing has been performed are executed.
